(19) United States
(12) Patent Application Publication
(10) Pub. No.: US 2022/0080070 A1
(43) Pub. Date: Mar. 17, 2022

(54) APPARATUS AND METHODS FOR TRIOXIDANE DISINFECTION

(71) Applicant: DD SterOZone LLC, Edina, MN (US)

(72) Inventors: Kevin FROST, Columbus, OH (US); Landon ADLER, Jacksonville, FL (US)

(21) Appl. No.: 17/474,748

(22) Filed: Sep. 14, 2021

Related U.S. Application Data
(60) Provisional application No. 63/078,469, filed on Sep. 15, 2020.

(51) Int. Cl. A61L 2/22; A61L 2/26; C01B 13/32; A01N 59/00

(52) U.S. Cl. CPC A61L 2/22; A61L 2/26; A61L 2202/15; A01N 59/00; A61L 2202/11; C01B 13/326

(57) ABSTRACT

Compositions, methods and apparatus for trioxidane disinfection. The compositions may contain trioxidane in microbicidal concentrations of trioxidane effective for disinfection of surfaces. The apparatus may be used to produce the compositions. The apparatus may be used to deliver the compositions. The apparatus may be used to perform one or more steps of the methods. The methods may include methods for disinfection with the trioxidane compositions produced in or delivered via one or more of the apparatus.

### CROSS-REFERENCE TO RELATED APPLICATION

This application claims the benefit of priority from U.S. Provisional Application No. 63/078,469, filed Sep. 15, 2020, the contents of which are hereby incorporated herein by reference in their entirety.

### FIELD OF THE TECHNOLOGY

Aspects of the disclosure relate to a trioxidane-containing composition, and its production and use for disinfection of surfaces. In particular, the disclosure relates to an apparatus within which the composition is produced and from which aqueous trioxidane of microbicidal concentration can be delivered to disinfect surfaces.

### BACKGROUND

Disinfection in medical settings has been advocated and, to various degrees of success, practiced from the times of Semmelweis and Lister in the mid-1800s when theories of communicable infection though microbiological agents were gaining acceptance. In the early 20^{th} Century, adoption of clinical aseptic and antiseptic practice was forced on the public by the 1918-1920 Spanish H1N1 flu pandemic. As a result, disinfection made its way into people's mindsets, homes and workplaces. The means for such disinfection were generally limited to basics still practiced today: Mechanical barriers such as gloves and face masks; and readily available chemical agents such as soap, alcohol and dilute bleach.

Those classical means of disinfection have been augmented in the last several decades, at least in medical settings, with ultraviolet (UV) and ozone techniques. UV light of particular wavelengths can disrupt bonds in bacteria and viruses, causing irreparable damage to the microbes. Ozone, a triatomic version of oxygen, designated O₃ (while familiar breath-of-life oxygen gas is diatomic and is designated O₂), is highly reactive and readily damages microbes' structural integrity.

Another reactive agent, hydrogen peroxide (designated H₂O₂), has a similar potent microbial disinfection effect in higher concentration than pharmacy grade's approx. 3%, but can be damaging to surfaces. Likewise, ultraviolet and ozone techniques each has disadvantages that may weigh against its advantages as a disinfection modality. Ultraviolet and ozone techniques both present hazards to unprotected humans and typically require exposure times of several tens of minutes to several hours. Additionally, UV techniques are ineffective for shadowed surfaces not accessible to the light, and ozone techniques require post-treatment removal of the O₃ gas) (by electronic/photonic processing and/or fanning/pumping, or the like).

The 21^{st} Century has seen SARS, MERS and COVID-19 intensifying attention on disinfection techniques for surfaces in medical, residential, educational, commercial and other settings. The 21^{st} Century has also brought into scientific focus a highly effective microbicidal disinfection agent, trioxidane (designated H₂O₃), which may be produced by reaction of ozone and hydrogen peroxide.

Mixtures of ozone and hydrogen peroxide have been used effectively for several decades in industrial applications for breaking down organic pollutants. Those applications include groundwater remediation because the chemical residue from mixtures of ozone and hydrogen peroxide are water, oxygen and other mainly innocuous environment-neutral compounds. The reaction of ozone and hydrogen peroxide that produces the relatively short-lived trioxidane as an intermediate and, subsequently, the innocuous breakdown compounds is termed the “peroxone reaction.”

Immune-system antibodies, in their strategic attack on microbial invaders, are thought to utilize the peroxone reaction on a scale limited to a few molecules of ozone and hydrogen peroxide. The small quantity of trioxidane product serves as the antibodies' lethal anti-microbial payload, indicative of the microbicidal effectiveness of trioxidane.

The peroxone reaction has been harnessed in surface disinfection for the reaction's potent microbicidal properties and clean breakdown products. Current techniques, however, involve sealing a room to be disinfected; filling the room with O₃ and with H₂O₂-containing water vapor/mist; and allowing time for molecules of the reactants to encounter each other within the room's volume, and react to form trioxidane. The short-lived trioxidane must be frequently replenished, necessitating lengthy periods of refilling the room with O₃ and with H₂O₂-containing water vapor/mist. Additionally, these techniques still require post-treatment removal of unreacted ozone, as well as subsequent unsealing of the room and wiping down of surfaces wet with the hydrogen peroxide vapor/mist.

Accordingly, it would be desirable to provide disinfection compositions, methods of using the compositions, and apparatus for producing and applying the compositions, that utilize the peroxone reaction without disadvantages of current techniques.

It would be further desirable, particularly during times of growing pandemic concerns, to provide compositions, methods and apparatus for trioxidane disinfection that are straightforward to implement, simple to utilize, and safe for users, clients and the environment.

### BRIEF SUMMARY

Compositions, methods and apparatus for trioxidane disinfection. The compositions may contain trioxidane in microbicidal concentrations of trioxidane effective for disinfection of surfaces. The apparatus may be used to produce the compositions. The apparatus may be used to deliver the compositions. The apparatus may be used to perform one or more steps of the methods. The methods may include methods for disinfection with the trioxidane compositions produced in or delivered via one or more of the apparatus.

In accordance with one aspect of the disclosure, provided is a mixture of chemical compounds for trioxidane disinfection of surfaces. The mixture typically comprises water; and in the water diatomic molecular oxygen; hydrogen peroxide in a range of about 0.5% to about 12% (wt/wt); triatomic molecular ozone in a range of about 1 ppm to about 100 ppm; and trioxidane formed by reaction of the ozone with the hydrogen peroxide, wherein the trioxidane is present in the mixture in a microbicidal concentration.

According to another aspect of the disclosure, a method is provided for producing trioxidane for disinfection. The method typically comprises applying energy to oxygenated water in a vessel, the energy configured to generate triatomic molecular ozone from diatomic oxygen in the water; in the water: generating the ozone; and reacting the ozone with hydrogen peroxide to yield trioxidane; and, within a preselected number of half-lives of the trioxidane after the applying, re-applying the energy to the water.

In accordance with a further aspect, provides is an apparatus for surface disinfection. The apparatus typically comprises a vessel configured to receive, diatomic oxygen in water; and hydrogen peroxide in a range, in the water, of about 0.5% to about 12% (wt/wt); an agitator configured to agitate the water and hydrogen peroxide; an energy source configured to produce ozone from the oxygen; and an ejector configured to transfer trioxidane produced in the vessel to an exterior of the vessel.

According to yet another aspect, provided is a sprayer for spraying a trioxidane-containing disinfecting mist, the sprayer comprising a sprayer nozzle configured to: receive an aqueous mixture of hydrogen peroxide, ozone and trioxidane; reduce a concentration of the ozone; and discharge a mist of droplets that include a microbicidal concentration of trioxidane.

### BRIEF DESCRIPTION OF THE DRAWINGS

The objects and advantages of the disclosure will be apparent upon consideration of the following detailed description, taken in conjunction with the accompanying drawings, in which like reference characters refer to like parts throughout, and in which:

FIG. 1 shows an illustrative apparatus for production of trioxidane in accordance with the disclosure;

FIG. 2 shows an illustrative apparatus for spraying trioxidane in accordance with the disclosure; and

FIG. 3 shows a flowchart of illustrative steps for trioxidane disinfection in accordance with the disclosure.

### DETAILED DESCRIPTION

Compositions, apparatus and methods for trioxidane disinfection are provided. The compositions may include microbicidal concentrations of trioxidane effective for disinfection of surfaces. The apparatus may be used to produce the compositions. The apparatus may be used to perform one or more steps of the methods. The methods may include methods for disinfection with trioxidane produced in or delivered via one or more of the apparatus.

Exemplary embodiments are shown and described below. Features, including structures, materials, volumes, functions and other attributes that are shown and described in connection with any of the embodiments may be combined, in whole or in part, with each other or included, in whole or in part, in other embodiments.

The composition may include a mixture of chemical compounds for trioxidane disinfection of surfaces. The mixture may include water in which are molecules of diatomic oxygen, O₂. The mixture may include molecules of hydrogen peroxide, H₂O₂, in the water. The hydrogen peroxide may be present in the mixture in a wt/wt percentage ranging from about 0.5% to about 12%. The mixture may include molecules of triatomic oxygen, ozone, O₃, in the water. The ozone may be present in the mixture at levels ranging from about 1 ppm to about 100 ppm. The mixture may contain molecules of trioxidane, H₂O₃, in the water. The trioxidane may be formed by reaction of the ozone with the hydrogen peroxide. The trioxidane may be present in the mixture in a microbicidal concentration effective for disinfection of surfaces.

The mixture may include a surfactant agent in the water. The surfactant agent may be present in the mixture in a wt/wt percentage ranging from about 0.5% to about 5%. The surfactant agent may be present in the mixture in a wt/wt percentage ranging from about 2% to about 3%.

At least some of the mixture may include a mist component. At least some of the mixture may include a bulk liquid component. The mixture may be agitated by an agitator. The agitator may agitate the mist component of the mixture. The agitator may mix the mist component of the mixture. The agitator may agitate the bulk liquid component of the mixture. The agitator may mix the bulk liquid component of the mixture. The agitator may transfer a fraction of the bulk liquid component into the mist component. The agitator may transfer a fraction of the mist component into the bulk liquid component of the mixture.

In the mixture, a portion of the diatomic oxygen may be disposed in the bulk liquid component of the mixture. In the mixture, a portion of the diatomic oxygen may be disposed in the mist component of the mixture.

The hydrogen peroxide may be present in the mixture in a wt/wt percentage ranging from about 0.5% to about 8%. The hydrogen peroxide may be present in the mixture in a wt/wt percentage ranging from about 6% to about 12%.

The ozone may be produced through exposure of the diatomic oxygen disposed in the mist component of the mixture, to an output of an energy source. The ozone may be produced through exposure of the diatomic oxygen disposed in the bulk liquid component of the mixture, to an output of an energy source.

The energy source may generate an electrostatic discharge. The energy source may generate a coronal discharge. The energy source may generate radiant energy. The energy source may generate far-ultraviolet light.

Concentration of the ozone in the mixture may range from about 1 ppm to about 60 ppm.

The concentration of the ozone in the mixture may range from about 40 ppm to about 100 ppm.

In the mixture, a bulk liquid component of the mixture and a mist component of the mixture may each be at least partially confined within a vessel. The vessel may encompass an energy output section of an energy source. The energy output section may be configured to expose the oxygen to energy suitable to produce the ozone from the oxygen.

The vessel may include an outlet that, when unobstructed, disposes at least a part of the mixture in fluid communication with an exterior of the vessel. The part of the mixture may be released via the outlet. The part of the mixture released may be released into a space exterior to the vessel. The part of the mixture released may have microbicidal properties. The microbicidal properties may be conferred by a trioxidane concentration in the part of the mixture released.

The methods may include a method for producing trioxidane for disinfection. The method may include applying energy to oxygenated water in a vessel. The energy may be configured to generate triatomic molecular ozone from diatomic oxygen in the water. The method may include generating ozone in the water. The method may include reacting the ozone with hydrogen peroxide in the water to yield trioxidane.

The method may include, within a preselected number of half-lives of the trioxidane after the applying, re-applying the energy to the water. The preselected number of half-lives may be fifteen. The preselected number of half-lives may be ten. The preselected number of half-lives may be five. The preselected number of half-lives may be one. The half-lives may be half-lives of trioxidane under conditions within the vessel.

The method may include supplying the energy from an energy source. The energy source may produce an electrostatic discharge. The energy source may produce radiant energy. The radiant energy may include light in the far-ultraviolet region.

In the method, at least a fraction of the water may be in a vaporous state. In the method, at least a fraction of the water may be contained in a mist.

The methods may include a method of disinfecting a surface. The method may include applying aqueous trioxidane mist to the surface. The surface may be exterior to a vessel in which the trioxidane is produced.

The method may include, within a preselected number of half-lives of the trioxidane after the applying, re-applying aqueous trioxidane mist to the surface. The preselected number of half-lives may be fifteen. The preselected number of half-lives may be ten. The preselected number of half-lives may be five. The preselected number of half-lives may be one. The half-lives may be half-lives of trioxidane under conditions exterior to the vessel. The half-lives may be half-lives of trioxidane under conditions upon the surface.

In the method, the mist may include a surfactant agent. In the method, the mist may include droplets bearing electrostatic charge.

The method may include allowing breakdown products of the mist to evaporate from the surface. The method may include the surface to dry.

The apparatus may include, and the methods may involve, an apparatus for surface disinfection. The apparatus may include a vessel. The vessel may be configured to receive diatomic oxygen in water. The water may be oxygen-enriched by addition of diatomic oxygen.

The vessel may be configured to receive hydrogen peroxide in the water. The hydrogen peroxide may be present in the water in a range of about 0.5% to about 12% (wt/wt). The range may be about 1% to about 7% (wt/wt).

The apparatus may include an agitator. The agitator may be configured to agitate the water. The agitator may be configured to agitate the oxygen. The agitator may be configured to agitate the hydrogen peroxide. The agitator may be configured to agitate the water and chemical species in the water. The agitator may produce a mist of a mixture that includes the water and the chemical species in the water.

The apparatus may include an energy source. The energy source may be configured to produce ozone from the oxygen. The energy source may be configured to produce about 1 ppm to about 100 ppm ozone in the water.

The energy source may produce an electrostatic discharge. The energy source may produce radiant energy. The radiant energy may include light in the far-ultraviolet region.

The apparatus may include an ejector. The ejector may be configured to transfer trioxidane produced in the vessel to an exterior of the vessel. The ejector may include an outlet for fluid transfer from the vessel. The ejector may include a sprayer in fluid communication with the outlet. The sprayer may be configured to spray an aqueous mist. The aqueous mist may contain a microbicidal concentration of trioxidane. Some of the trioxidane in the mist may be produced within the sprayer by reaction of ozone and hydrogen peroxide within the sprayer.

The apparatus may include, and the methods may involve, a sprayer for spraying a trioxidane-containing disinfecting mist. The sprayer may include a sprayer nozzle. The sprayer nozzle may be configured to receive an aqueous mixture of hydrogen peroxide, ozone and trioxidane. The sprayer nozzle may be configured to reduce a concentration of the ozone. The sprayer nozzle may be configured to discharge a mist of droplets. The droplets may include a microbicidal concentration of trioxidane.

The sprayer may be an electrostatic sprayer. The droplets may bear an electrostatic charge.

The mist may include droplets of micron range size. The micron range may be from about 1 micron to about 100 microns. The micron range may be from about 1 micron to about 60 microns. The micron range may be from about 40 microns to about 100 microns.

The sprayer nozzle may be configured to reduce the concentration of ozone by application of radiant energy. The radiant energy may include ultraviolet (UV) light. The UV light may include near-UV bands. The radiant energy may include visible light. The visible light may include green bands.

The sprayer nozzle may be configured to receive the mixture with the mixture being at least partly driven by pressure. The pressure may be provided from within a vessel in fluid communication with the sprayer. In the vessel, trioxidane may be produced by reaction of ozone and hydrogen peroxide.

Apparatus and methods described herein are illustrative. Apparatus and methods in accordance with this disclosure will now be described in connection with the figures, which form a part hereof. The figures show illustrative features of apparatus and method steps in accordance with the principles of this disclosure. It is understood that other embodiments may be utilized, and that structural, functional, and procedural modifications may be made without departing from the scope and spirit of the present disclosure.

FIG. 1 shows apparatus 100 for production and delivery of trioxidane compositions for disinfection. Apparatus 100 includes vessel 101. Vessel 101 is configured to contain mixture 103. Mixture 103 includes solvent species and solute species (not individually shown); the solvent species include water and the solute species include diatomic molecular oxygen, triatomic molecular oxygen, hydrogen peroxide and trioxidane.

Mixture 103 has a bulk liquid component 105. Mixture 103 has a mist component 107.

Apparatus 100 includes coronal discharge electrodes 113 and coronal discharge control 115. Additionally, optionally or alternatively, apparatus 100 includes light source 117, UV-transparent shroud 119 and light source control 121.

Apparatus 100 includes agitator 109.

In operation of apparatus 100, agitator 109 agitates and mixes mixture 103. Agitator 109 is configured to transfer a fraction of bulk liquid component 105 to mist component 107. Agitator 109 is configured to transfer a fraction of mist component 107 to bulk liquid component 105.

In operation of apparatus 100, control 115 charges electrodes 113, building a voltage difference between electrodes 113 until coronal (electrostatic) discharge 111 is generated. Discharge 111 produces triatomic ozone from diatomic oxygen, as given be 3O₂→2O₃. Additionally, optionally or alternatively, far-UV light emitted by light source 117, produces triatomic ozone from diatomic oxygen.

The peroxone reaction (not shown) of the hydrogen peroxide and the ozone produces trioxidane.

Apparatus 100 includes ejector 123. Ejector 123 is shown closed by valve 125. For delivery of part of trioxidane-containing mist component 107 to a surface exterior vessel 101, valve 125 would be open (not shown). The delivery through ejector 123 may be pressure-driven. Fan 127 is configured to provide air-assist pressure to drive the part of trioxidane-containing mist component 107 through ejector 123.

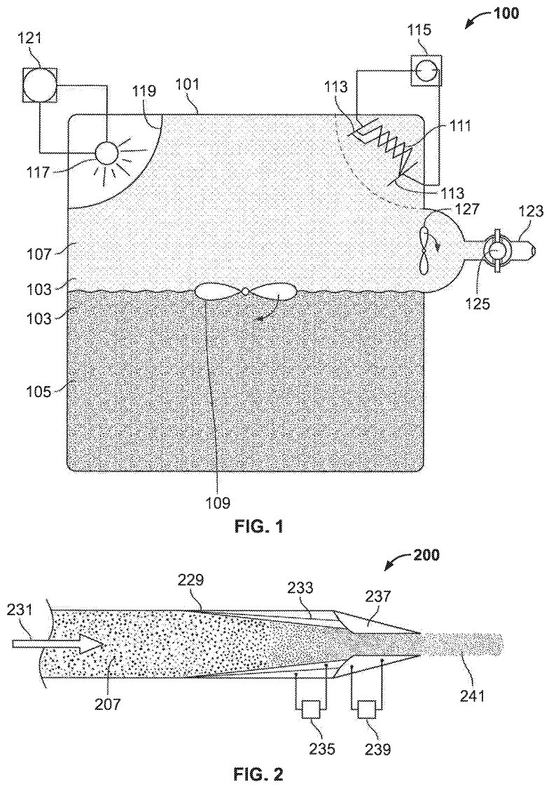

FIG. 2 shows sprayer 200. Sprayer 200 may be in fluid communication with ejector 123 with valve 125 open (both shown in FIG. 1). Sprayer 200 is depicted in operation, with air-assist 231 driving mist component 207 into and through sprayer 200. Air-assist 231 may be driven by fan 127 (shown in FIG. 1).

Sprayer 200 includes sprayer nozzle 229. Nozzle 229 includes electrostatic plates 233 and electrostatic control 235. Control 235 is configured, via plates 233, to charge droplets of incoming mist component 207, producing uniformly small droplets of micron range size.

Nozzle 229 includes light source 237, controlled by light source control 239. Light source 237 is configured to produce light of wavelengths disruptive of internal bonds of ozone molecules, thus reducing an ozone concentration within droplets of mist 241 exiting from sprayer 200.

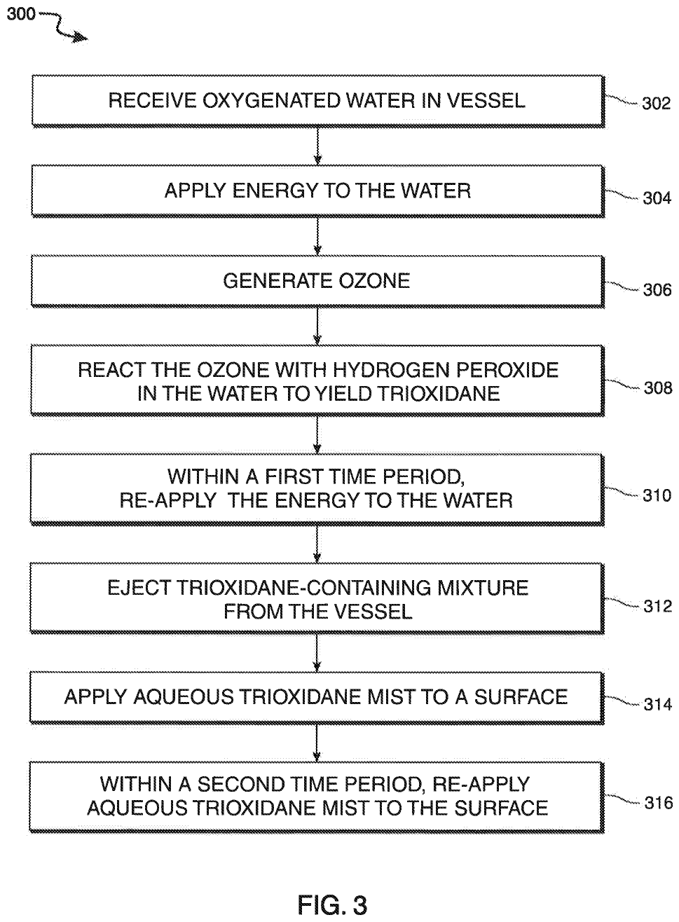

FIG. 3 is a flowchart of method steps of a trioxidane disinfection process. The process may begin at step 302.

At step 302, oxygenated water may be received in a vessel. The vessel may be vessel 101 of apparatus 100 (shown in FIG. 1).

The process may continue to step 304. At step 304, energy is applied to the water, to bring the process forward to step 306, at which step ozone is generated. The ozone may be generated from diatomic oxygen in the water by application of the energy (as discussed above regarding coronal discharge 111 and light source 117 shown in FIG. 1).

At step 308, the ozone generated at step 306 may react with hydrogen peroxide in the water to yield trioxidane. The trioxidane, produced via the peroxone reaction, is short-lived. To maintain a microbicidal concentration of trioxidane levels, the process may proceed to step 310.

At step 310, the energy is re-applied to the water within a first preselected time period. The first preselected time period may be a multiple of a half-life of trioxidane under conditions in the vessel. Re-application of the energy may generate more ozone to react, via the peroxone reaction, with hydrogen peroxide in the water. This may maintain a microbicidal trioxidane concentration in the mixture of water O₂, O₃, H₂O₂ and trioxidane in the vessel.

At step 312, a part of the mixture may be ejected from the vessel. Ejection of the mixture may be facilitated by ejector 123 with valve 125 open, and further assisted by fan 127 (all three shown in FIG. 1).

As discussed above regarding sprayer 200 (shown in FIG. 2), ejector 123 (shown in FIG. 1) may be in fluid communication with sprayer 200. This fluid communication may facilitate step 314. At step 314, aqueous trioxidane mist (such as mist 241 shown in FIG. 2), may be applied to a surface to be disinfected.

At step 316, aqueous trioxidane mist may be re-applied to the surface within a second preselected time period. The second preselected time period may be a multiple of a half-life of trioxidane under conditions on the surface. Re-application of the aqueous trioxidane mist may maintain a microbicidal trioxidane concentration on the surface.

It should be noted that the foregoing shows and/or describes exemplary embodiments of compositions, methods and apparatus according to the invention.

The steps of methods may be performed in an order other than the order shown and/or described herein. Embodiments may omit steps shown and/or described in connection with illustrative methods. Embodiments may include steps that are neither shown nor described in connection with illustrative methods.

Illustrative method steps may be combined. For example, an illustrative method may include steps shown in connection with another illustrative method.

Apparatus may omit features shown and/or described in connection with illustrative apparatus. Embodiments may include features that are neither shown nor described in connection with the illustrative apparatus. Features of illustrative apparatus may be combined. For example, an illustrative embodiment may include features shown in connection with another illustrative embodiment.

The drawings show illustrative features of compositions, methods and/or apparatus in accordance with the principles of the invention. The features are illustrated in the context of selected embodiments. It will be understood that features shown in connection with one of the embodiments may be practiced in accordance with the principles of the invention along with features shown in connection with another of the embodiments.

One of ordinary skill in the art will appreciate that the steps shown and described herein may be performed in other than the recited order and that one or more steps illustrated may be optional. The methods of the above-referenced embodiments may involve the use of any suitable elements, steps, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

Thus, compositions, methods and apparatus for trioxidane disinfection are provided. Persons skilled in the art will appreciate that the present invention can be practiced by other than the described embodiments, which are presented for purposes of illustration rather than of limitation, and that the present invention is illustrated also by the following exemplary embodiments.

What is claimed is:

1. A mixture of chemical compounds for trioxidane disinfection of surfaces, the mixture comprising:
water; and,
in the water:
diatomic molecular oxygen;
hydrogen peroxide in a range of about 0.5% to about 12% (wt/wt);
triatomic molecular ozone in a range of about 1 ppm to about 100 ppm; and
trioxidane formed by reaction of the ozone with the hydrogen peroxide, wherein the trioxidane is present in the mixture in a microbicidal concentration.

2. The mixture of claim 1 further comprising a surfactant agent in the water in an amount of about 0.5% to about 5% (wt/wt).

3. The mixture of claim 1, wherein at least some of the mixture comprises a mist component of the mixture.

4. The mixture of claim 1, wherein a portion of the oxygen is disposed in a bulk liquid component of the mixture.

5. The mixture of claim 1, wherein the hydrogen peroxide range is about 0.5% to 12% (wt/wt).

6. The mixture of claim 1, wherein the ozone range is about 1 ppm to about 100 ppm.

7. A method for producing trioxidane for disinfection, the method comprising:
applying energy to oxygenated water in a vessel, the energy adapted to generate triatomic molecular ozone from diatomic oxygen in the water;
in the water:
generating the ozone; and
reacting the ozone with hydrogen peroxide to yield trioxidane; and,
within a preselected number of half-lives of the trioxidane after the applying, re-applying the energy to the water.

8. The method of claim 7, wherein the energy source produces an electrostatic discharge, radiant energy, or a combination thereof.

9. The method of claim 7, wherein the preselected number of half-lives is from five to fifteen.

10. A method of disinfecting a surface, the method comprising:
applying the mixture of chemical compounds of claim 1 to a surface; and
within a preselected number of half-lives of the trioxidane after the applying, re-applying aqueous trioxidane mist to the surface.

11. The method of claim 10 further comprising allowing the surface to dry.

12. An apparatus for surface disinfection, the apparatus comprising:
a vessel configured to receive:
diatomic oxygen in water; and
hydrogen peroxide in a range, in the water, of about 0.5% to about 12% (wt/wt);
an agitator configured to agitate the water and hydrogen peroxide;
an energy source configured to produce ozone from the oxygen; and
an ejector configured to transfer trioxidane produced in the vessel to an exterior of the vessel.

13. The apparatus of claim 12, wherein the water is oxygen-enriched by addition of diatomic oxygen.

14. The apparatus of claim 12, wherein the agitator transfers a fraction of the bulk liquid component into the mist component.

15. The apparatus of claim 12, wherein the agitator produces a mist of a mixture that includes the water and chemical species in the water.

16. The apparatus of claim 12, wherein the agitator transfers a fraction of the mist component into a bulk liquid component of the mixture.

17. The apparatus of claim 12, wherein the energy source produces an electrostatic discharge, a coronal discharge, radiant energy, or a combination thereof.

18. The apparatus of claim 12, wherein the energy source is configured to produce about 1 ppm to about 100 ppm ozone in the water.

19. The apparatus of claim 12, wherein the ejector includes:
an outlet for fluid transfer from the vessel; and
a sprayer in fluid communication with the outlet, the sprayer configured to spray an aqueous mist containing a microbicidal concentration of trioxidane.

20. The apparatus of claim 12, wherein the sprayer comprises:
a sprayer nozzle configured to:
receive an aqueous mixture of hydrogen peroxide, ozone and trioxidane from the vessel;
reduce a concentration of the ozone; and
discharge the mist of droplets containing the microbicidal concentration of trioxidane.
